(19) United States
(12) Patent Application Publication
(10) Pub. No.: US 2025/0101519 A1
(43) Pub. Date: Mar. 27, 2025

(54) USE OF HYPOXIA-INDUCIBLE FACTOR 1 ALPHA AS MARKER IN DEPRESSION RECURRENCE DIAGNOSIS

(71) Applicant: NINGBO UNIVERSITY, Ningbo, Zhejiang (CN)

(72) Inventors: Chuang WANG, Ningbo, Zhejiang (CN); Jia XU, Ningbo, Zhejiang (CN)

(21) Appl. No.: 18/833,488

(22) PCT Filed: Jan. 18, 2023
(86) PCT No.: PCT/CN2023/072984

§ 371 Date: Jul. 26, 2024

(30) Foreign Application Priority Data
Jan. 27, 2022 (CN) 202210102924.7

(51) Int. Cl. C12Q 1/6883; G01N 33/68; G16H 50/30; G16H 50/70

(52) U.S. Cl. CPC C12Q 1/6883; G01N 33/6893; G16H 50/30; G16H 50/70; C12Q 2600/118; C12Q 2600/158; G01N 2333/4706; G01N 2800/304; G01N 2800/54

(57) ABSTRACT

Hypoxia-inducible factor 1α protein or a detection reagent thereof can be used in depression recurrence diagnosis. It has been demonstrated that the specificity of hypoxia inducible factor 1α protein increases in the serum of patients with depression recurrence after interventions. As such, hypoxia inducible factor 1α protein can be used as a peripheral blood biomarker for the early screening or differentia diagnosis of depression recurrence, and for post-intervention assessment of treatment efficacy of patients with depression.

### TECHNICAL FIELD

The present invention belongs to the field of biological science and relates to rapid, convenient, and specific early screening or differential diagnosis of depression recurrence. Specifically, it relates to use of hypoxia-inducible factor 1 alpha as marker in early screening or differential diagnosis of depression recurrence.

### BACKGROUND

Major depressive disorder (MDD) is a major mental illness that is influenced by the interaction between genes and the environment. Its main clinical features are significant and persistent low mood and decreased interest. In severe cases, there may even be suicidal tendencies and a significant risk of disability. According to epidemiological data compiled by the World Health Organization in 2019, over 350 million people worldwide suffered from depression, and the 12-month prevalence rate of depression in China was 3.6%, while the lifelong prevalence rate reached 6.9%. The prevention and treatment of depression is urgent.

At present, the main difficulties faced in the diagnosis and treatment of depression are: \textcircled{1} slow onset of antidepressant drugs; \textcircled{2} low medication adherence; \textcircled{3} slow development of new drugs; \textcircled{4} high recurrence rate, etc. Especially prone to recurrence has become a key and difficult point for the worsening of depressive symptoms and the prevention and treatment of depression. Research has shown that 34-83% of patients with severe depression experience new episodes of depression within 6 months. It is worth noting that 60% of depression patients are at risk of developing new depression episodes after their first episode, while those who have experienced two or more episodes have a high probability of recurrence, ranging from 70% to 90%. Long term recurrence of chronic depression leads to depression becoming a major chronic mental illness, which poses difficulties for the prevention and treatment of depression.

In clinical practice, the diagnosis of depression recurrence is mainly based on the patient's detailed medical history and psychological symptoms, and is comprehensively judged through subjective scores such as the Hamilton Depression Scale (HAMD) and the Montgomery-Asberg Depression Rating Scale (MADRS). Due to the different subjective experiences of physicians, the diagnostic results also vary. Therefore, in order to make the diagnosis of depression recurrence more objective, reduce human factors, improve the consistency and accuracy of diagnosis, and achieve early diagnosis, detection, and intervention of depression recurrence, the development of effective objective biomarkers and the establishment of effective detection methods are of great significance for patients with depression. Therefore, how to specifically diagnose the recurrence of depression and provide effective treatment has become a very urgent issue in the current clinical diagnosis and treatment of depression. Peripheral blood of patients with depression is easily obtained, and it is minimally invasive for patients, and inexpensive. It is easy to achieve automate detection and can effectively assist uncertain human factors in current diagnosis, thereby reducing misdiagnosis rates. This biomarker has good sensitivity and specificity.

The use of molecular biology detection methods to scientifically detect the potential risk of recurrence in patients with depression has important clinical significance and social value. Therefore, there is an urgent need in this field to develop highly sensitive and specific biomarkers for clinical diagnosis and treatment of depression recurrence, to address the difficulties in the diagnosis of depression recurrence, and there is also a need to develop a detection system for predicting and/or diagnosing the risk of depression recurrence, to improve the objectivity and accuracy of depression recurrence assessment.

### SUMMARY OF THE INVENTION

The purpose of the present invention is to provide a specific biomarker hypoxia-inducible factor 1 alpha with high sensitivity and specificity for predicting and/or diagnosing the risk of depression recurrence and use thereof.

In the first aspect of the present invention, it provides a use of a protein of depression recurrence risk marker, or a detection reagent thereof in the preparation of a diagnostic reagent or kit for the evaluation of the risk of depression recurrence; wherein, the depression recurrence risk marker includes HIF-1α protein.

In another preferred embodiment, the diagnostic reagent or kit is used to detect the level of the risk marker in a tested sample.

In another preferred embodiment, the tested sample is selected from the group consisting of blood, plasma, serum, and a combination thereof.

In another preferred embodiment, the expression level of the risk marker includes the expression level in blood, plasma, or serum.

In another preferred embodiment, the evaluation includes early diagnosis, auxiliary diagnosis, or a combination thereof.

In another preferred embodiment, the gene or protein of the risk marker is derived from humans.

In another preferred embodiment, the detection reagent is coupled with or carries detectable markers.

In another preferred embodiment, the detectable markers are selected from the group consisting of: chromophores, chemiluminescent groups, fluorophores, isotopes, and enzymes.

In another preferred embodiment, the diagnostic reagent includes antibodies, primers, probes, sequencing libraries, nucleic acid chips (such as DNA chips), or protein chips.

In another preferred embodiment, the nucleic acid chip comprises a substrate and a specific oligonucleotide probe spotted on the substrate, wherein the specific oligonucleotide probe comprises a probe specifically bound to the polynucleotide (mRNA or cDNA) of the risk marker.

In another preferred embodiment, the detection reagent comprises a reagent for detecting the content or activity of HIF-1α protein using enzyme-linked immunosorbent assay.

In another preferred embodiment, the reagent for detecting HIF-1α protein content using enzyme-linked immunosorbent assay is a HIF-1α protein content enzyme-linked immunosorbent assay kit.

In another preferred embodiment, the reagent for detecting the content of HIF-1α protein using enzyme-linked immunosorbent assay is an ELISA kit.

In another preferred embodiment, the ELISA kit comprises: HIF1A (Human) ELISA Kit (RAB0507, Abnova), and Human HIF-1 alpha ELISA Kit (ab171577, Abcam).

In another preferred embodiment, the depression recurrence risk marker further comprises a gene, transcript, or protein of one or more markers selected from the group B consisting of (B1) HSP90 and (B2) BICC1.

In another preferred embodiment, the diagnostic reagent or kit is used to detect the protein level of HIF-1α and the transcript level or protein quantity or activity of HSP90 in the tested sample.

In another preferred embodiment, the diagnostic reagent or kit is used to detect the protein level of HIF-1α and the transcript level or protein quantity or activity of HSP90 and BICC1 in the tested sample.

In another preferred embodiment, when the expression level C1 of the risk marker HIF-1α protein of a subject is significantly higher than the reference value C0, it indicates that the subject has a high risk of depression recurrence.

In another preferred embodiment, the diagnostic reagent or kit is also used to detect the mRNA level of HIF-1α in the tested sample.

In another preferred embodiment, if (a) the protein quantity or activity of HIF-1α increases but the mRNA level of HIF-1α does not show significant changes (or does not change) in a subject, while (b) the protein quantity or activity or mRNA level of HSP90 increases, it indicates that the subject has an increased risk of depression recurrence (an increased risk of depression recurrence associated with reduced degradation of HIF-1α protein).

In another preferred embodiment, the kit further comprises a label or a specification, wherein the label or the specification indicates that the kit is used for (a) diagnosing the risk of depression recurrence, and/or (b) evaluating the therapeutic effect of depression recurrence.

In another preferred embodiment, the label or the specification indicates the following content: if the transcription levels of HSP90 and BICC1 of the risk markers in a subject are significantly higher than the reference value, it indicates that the subject has a higher risk of depression recurrence than general depression patients.

In another preferred embodiment, if the transcription levels of HSP90 and BICC1 of the risk markers in a subject are not higher than the reference value, it indicates that the subject has a low risk of depression recurrence.

In another preferred embodiment, the label or the specification indicates the following content: if the translation level of the risk marker in the subject is significantly higher than the reference value, it indicates that the risk of depression recurrence in the subject is higher than that of general depression patients.

In another preferred embodiment, if the HIF-1α protein level in a subject is not higher than the reference value, it indicates that the subject has a low risk of depression recurrence.

In another preferred embodiment, the label or the specification indicates the following content: if the concentration of the risk marker (such as HIF-1α) C1 in a subject is significantly higher than the reference value C0, then the subject has a higher probability of depression recurrence than general depression patients.

In another preferred embodiment, the reference value C0 represents the concentration of the risk marker in the same sample from a normal population or patients without depression recurrence.

In another preferred embodiment, the term “significantly higher” refers to a ratio of C1/C0≥1.5, preferably ≥2, and more preferably ≥3.

In another preferred embodiment, if the protein quantity or activity of HIF-1α increases in the tested sample of a subject, it indicates that the subject has an increased risk of depression recurrence.

In another preferred embodiment, if the protein quantity, activity, or mRNA level of HSP90 increases in the tested sample of a subject, it indicates that the subject has an increased risk of depression recurrence.

In another preferred embodiment, if (a) the protein quantity or activity of HIF-1α increases in the tested sample of a subject, and (b) the protein quantity or activity or mRNA level of HSP90 increases, it indicates that the subject has an increased risk of depression recurrence.

In another preferred embodiment, if (a) the protein quantity or activity or mRNA level of BICC1 increases in the tested sample of a subject, and (b) the protein quantity or activity of HIF-1α increases, it indicates that the subject has an increased risk of depression recurrence.

In another preferred embodiment, if (a) the protein quantity or activity of HSP90, HIF-1α, and BICC1 increases in the tested sample of a subject, and/or (b) the mRNA levels of HSP90 and BICC1 increase, it indicates that the subject has an increased risk of depression recurrence.

In another preferred embodiment, the diagnostic reagent or kit is used to detect the level of the risk marker in the tested sample.

In another preferred embodiment, the diagnostic reagent or kit comprises: monoclonal antibodies or polyclonal antibodies that bind to HIF-1α protein.

In another preferred embodiment, the tested sample is derived from a subject selected from the group consisting of: a subject without depression, a subject which is susceptible to depression, a patient with first-onset depression, a patient with depression recurrence, and a combination thereof.

In the second aspect of the present invention, it provides a depression recurrence risk assessment device, which comprises:
(a) an input module, which is configured for inputting depression recurrence risk marker data in blood, plasma, or serum of a subject;
wherein, the risk marker comprises HIF-1α protein;
(b) a data processing module, which is configured for processing depression recurrence risk marker data and providing recurrence risk assessment result, wherein the processing comprises: comparing the expression level C1 of the marker with the reference value C0, wherein, when C1 is significantly higher than C0, it indicates that the subject has a high risk of depression recurrence; when C1 is not significantly higher than C0, it indicates that the subject has a low risk of depression recurrence; and
(c) an output module, which is configured for outputting the assessment result.

In another preferred embodiment, the risk marker further comprises a gene, transcript, or protein of one or more markers selected from the group B consisting of (B1) HSP90 and (B2) BICC1.

In another preferred embodiment, the device further comprises a detection module, which is configured for detecting the protein level or activity of the risk marker.

In another preferred embodiment, the detection module is selected from the group consisting of: an ELISA analyzer, a PCR instrument, a sequencer, and a combination thereof.

In another preferred embodiment, the depression recurrence risk marker data comprises the protein quantity or activity data of HIF-1α and mRNA (or transcript) levels of HIF-1α.

In another preferred embodiment, the data processing module simultaneously evaluates the protein quantity and mRNA level of HIF-1α.

In another preferred embodiment, the processing comprises: when the protein quantity of HIF-1α is significantly increased in the tested sample of a subject, but the mRNA level of HIF-1α is not significantly increased (such as unchanged or substantially unchanged), it indicates that the subject has a high risk of depression recurrence.

In another preferred embodiment, if (a) the protein quantity or activity of HIF-1α increases but the mRNA level of HIF-1α does not show significant changes (or does not change) in the tested sample of a subject, while (b) the protein quantity or activity or mRNA level of HSP90 increases, it indicates that the subject has an increased risk of depression recurrence which is associated with reduced degradation of HIF-1α protein.

It should be understood that within the scope of the present invention, each technical feature of the present invention described above and in the following (such as examples) may be combined with each other to form a new or preferred technical solution, which is not listed here due to space limitations.

### BRIEF DESCRIPTION OF DRAWINGS

FIG. 1 shows the peripheral blood HIF-1α protein content change of the first-time major depression patient in Example 1 of the present invention, and the peripheral blood HIF-1α protein content change of recurrent and non-recurrent cases after drug treatment.

FIG. 2 shows the HAMD-17 scores and the correlation analysis of HIF-1α expression in peripheral blood of healthy and depressed subjects in Example 1 of the present invention.

FIG. 3 shows the HAMD-17 scores and the correlation analysis of HIF-1α expression in peripheral blood of healthy, recurrent and non-recurrent depression subjects after treatment in Example 1 of the present invention.

FIG. 4 shows the roadmap of the chronic unpredictable mild stress (CUMS) induced depression recurrence model in Example 2 of the present invention. Among them, the depression like behavior test was evaluated using the sugar preference test (SPT), open field test (OFT), novelty suppressed feeding test (NSFT), and forced swimming test (FST). The criteria for behavioral judgment and evaluation are as follows: (1) during the baseline period of the experiment, the inclusion criteria for animals are: the sugar preference range is 60%-90%, and the range of threading times during autonomous activities is 150-200 times/5 minutes. (2) After the first 5-week CUMS and the subsequent 5-week CUMS for modeling, the screening criteria for depressed and non-depressed animals are as follows: \textcircled{1} depressed mice: the sugar preference is <60%, and its decrease is >30%; \textcircled{2} non-depressed/normal control animals: the sugar preference range is still 60%-90%, and its decrease or increase is <30%.

FIG. 5 shows the expression and transcription evaluation of HSP90, HIF-1α, and BICC1 in the prefrontal cortex after depression recurrence in Example 2 of the present invention. RE-CUMS significantly induced an increase in HSP90, HIF-1α, and BICC1 protein expression. The transcription changes of hsp90 and bicc1 were significantly increased, while hif-1α did not show significant increase. The immunohistochemical study results showed that RE-CUMS significantly induced an increase in HSP90 and BICC1 labeled positive neurons.

### DETAILED DESCRIPTION

Through extensive and deep research, the inventor unexpectedly discovered a new risk marker for depression recurrence for the first time. The risk marker for depression recurrence comprises HIF-1α, and a method and a kit for predicting and/or evaluating the risk of depression recurrence were correspondingly developed. The use of HIF-1α alone, or in combination with other depression recurrence risk markers such as HSP90, BICC1, etc., can predict and/or diagnose the risk of depression recurrence with high sensitivity and specificity. On this basis, the present invention has been completed.

Experiments have shown that the depression recurrence risk markers or combinations thereof of the present invention can effectively warn or diagnose the potential risk of depression recurrence in patients. Using these depression recurrence risk markers to predict and/or detect the likelihood or disease status of depression recurrence in patients has high accuracy and specificity, providing a reference standard for early warning and/or diagnosis of depression recurrence in clinical practice.

The application of the detection system based on the depression recurrence markers of the present invention provides a relatively objective technical means for evaluating depression recurrence, which can effectively avoid diagnostic bias problems caused by scale analysis and subjective experience evaluation, and provides new detection methods for improving the objectivity and accuracy of depression recurrence assessment.

### Risk Marker for Depression Recurrence

In the present invention, the terms “the depression recurrence risk marker of the present invention”, “the recurrence risk marker of the present invention”, and “the risk marker of the present invention” can be used interchangeably, and all of them refer to the depression recurrence risk marker HIF-1α of the present invention.

In addition, additional markers that can be used in conjunction with the risk marker HIF-1α of the present invention include (but are not limited to): HSP90, BICC1.

In the present invention, the risk marker includes a gene (DNA), a cDNA, a protein, or a combination thereof.

The protein of the marker of the present invention may or may not contain the initial methionine. In addition, the term also includes a full-length depression recurrence risk marker protein and fragments thereof. In the present invention, the depression recurrence risk marker protein comprises its complete amino acid sequence, its secreted protein, its mutants, and its functionally active fragments.

HIF-1α is hypoxia inducible factor-1 alpha, gene name: HIF1AN (Homo sapiens), UniProtKB ID: Q9NWT6, NCBI Gene: 55662, which plays a role as the main transcriptional regulator of hypoxic adaptive response. Under hypoxic conditions, it activates the transcription of over 40 genes, including erythropoietin, glucose transporter, glycolytic enzymes, vascular endothelial growth factor, HILPDA, and other genes whose protein products that increase oxygen transport or promote metabolic adaptation to hypoxia. HIF-1α plays an important role in the pathophysiology of embryonic vascularization, tumor angiogenesis, and ischemic diseases.

HSP90 is heat shock protein 90, which includes an alpha subunit (gene name: HSP90AA1-Human, UniProtKB ID: P07900, NCBI Gene: 3326) and a beta subunit (gene name: HSP90AB1-Human, UniProtKB ID: P08238, NCBI Gene: 3326). HSP90 can promote the maturation, structural maintenance, and properly regulated molecular chaperones of a specific target protein, such as participating in cell cycle control and signal transduction. It experiences a functional cycle related to its ATPase activity, which is crucial for its chaperone protein activity. And it participates in the regulation of substrate recognition, ATPase cycle, and chaperone protein function.

BICC1 is Protein bicaudal C homolog 1, gene name: BICC1-Human, UniProtKB ID: Q9H694, NCBI Gene: 80114. BICC1, as a potential RNA binding protein, acts as a negative regulator of Wnt signaling and may be involved in regulating gene expression during embryonic development.

### CUMS Induced Depression Recurrence Model

The present invention also provides a model for constructing depression recurrence induced by chronic unpredictable mild stress (CUMS) and its screening scheme.

A preferred model construction and screening scheme may include the following steps:
Step 1: Baseline grouping (Round 0): after baseline testing, 150 male mice were divided into NC group (n=30) and CUMS group (n=120);
Step 2: SPT screening (Round 1), 150 animals were selected and divided into NC group (n=30) and CUMS group (n=120). After SPT screening, 129 animals were included for initial stress modeling and behavioral screening;
Step 3: First post-stress depression behavior test (Round 2), 129 animals were selected and divided into NC group (n=25) and CUMS group (n=104). After SPT, OFT, NSFT, and FST screening, 107 animals were included for Fluoxetine drug treatment;
Step 4: Evaluation of depression behavior after Fluoxetine drug treatment (Round 3), 107 animals were selected and divided into NC+Sal group (n=25), DE+Sal group (n=32), and DE+FLX group (n=50); after treatment with Fluoxetine, mice with depressive like behavior (n=107) were selected through SPT, OFT, NSFT, and FST screening. Drug elution was performed, followed by SPT screening of drug eluted animals. 87 animals were included for secondary stress (Re-exposed CUMS);
Step 5: Evaluation of recurrent depression behavior after secondary stress (Round 4), 87 animals were selected and divided into NC+Sal group (n=22), DE+Sal group (n=27), and DE+FLX group (n=38); after secondary stress, 50 animals were selected through SPT, OFT, NSFT, and FST for subsequent evaluation of mouse models of depression recurrence;
Step 6: Evaluation of HSP90, HIF-1a, BICC1, or a combination thereof in the brain of mice after depression recurrence (Round 5), 50 animals were selected and divided into NC group (n=17), RE-CUMS-DE group (n=18), and RE-CUMS-w/o DE group (n=15) for the evaluation of HSP90, HIF-1α, and BICC1 expression and transcription changes in the prefrontal cortex after depression recurrence.

### Detection Method

Based on the high expression of the depression recurrence risk marker HIF-1α in blood, plasma or serum, the present invention also provides a corresponding diagnostic method for depression recurrence.

The present invention relates to a diagnostic experiment method for quantitative and location detection of the gene or protein level of the depression recurrence risk marker HIF-1α. These experiments are well-known in this field. The gene and protein levels of the depression recurrence risk marker HIF-1α detected in the experiment can be used for the diagnosis (including auxiliary diagnosis) of the risk of depression recurrence.

One preferred method is to quantitatively detect the protein of depression recurrence risk marker.

Preferably, a method for detecting the presence of marker protein in a sample is to use a specific antibody for detection, which includes: contacting the sample with a specific antibody against the protein of the depression recurrence risk marker HIF-1α; observing whether an antibody complex is formed. The formation of antibody complex indicates the presence of protein of depression recurrence risk marker HIF-1α in the sample.

The depression recurrence risk marker protein or its polynucleotide can be used for the diagnosis of depression recurrence. Part or all of the polynucleotide of the present invention can be fixed as probes on a microarray or a DNA chip for differential expression analysis and gene diagnosis of genes in mononuclear cells. Antibodies against depression recurrence risk marker can be fixed on a protein chip to detect depression recurrence risk marker proteins in a sample.

Based on the research of the present invention, there is a significant increase in the protein level of HIF-1α in patients with depression recurrence. Therefore, HIF-1α can be used as a marker for detecting or diagnosing the risk of depression recurrence, especially in auxiliary and/or early diagnosis. When performing test, if the ratio of the expression level C1 of the marker gene (i.e., HIF-1α) to the corresponding expression level C0 in the normal population (C1/C0) is ≥1.5, preferably ≥2 or ≥3, it can be considered as an increased risk of depression recurrence.

In addition, the inventor also unexpectedly found that in a subject at high risk of depression recurrence, although the HIF-1α protein level in the tested sample increased (or significantly increased), the mRNA level of HIF-1α remained substantially unchanged or did not show significant differences. This suggests that after the expression of the HIF-1α protein in the present invention, its degradation rate is low or inhibited, leading to an increase in the final level of HIF-1α protein content. The study by the inventor suggests that the elevated HIF-1α protein without elevated mRNA level is often accompanied by an increase in HSP90 protein level, indicating a correlation between the decreased degradation of HIF-1α with HSP90 protein.

Therefore, in the present invention, quantitative detection (such as PCR or sequencing) of HIF-1α mRNA level can also be performed to identify this specific high-risk subtype of depression recurrence.

In the present invention, if the protein quantity or activity of HIF-1α increases but the mRNA level of HIF-1α does not significantly change (or does not change), and the protein quantity or activity or mRNA level of HSP90 protein increases, it indicates an increased risk of depression recurrence in the subject and is associated with a decrease in the degradation of HIF-1α protein.

### Kit

Based on the correlation between depression recurrence risk markers and the risk of depression recurrence, the depression recurrence risk marker HIF-1α can be used as a diagnostic marker for depression recurrence risk.

The present invention also provides a kit for diagnosing the risk of depression recurrence, comprising a detection reagent for detecting the depression recurrence risk marker HIF-1α gene, protein, or a combination thereof. Preferably, the kit comprises an antibody or immunoconjugate against the depression recurrence risk marker HIF-1α of the present invention, or an active fragment thereof; or primers or primer pairs for specifically amplifying the cDNA of the depression recurrence risk marker HIF-1α, probes, or chips.

In another preferred embodiment, the kit further comprises a label or a specification, wherein the label or the specification indicates that the kit is used for diagnosing the risk of depression recurrence and/or evaluating the therapeutic effect of depression recurrence.

The main advantages of the present invention include:
(a) The risk markers of the present invention can efficiently and accurately predict the risk of depression recurrence;
(b) The detection system of the present invention can provide accurate early warning and evaluate the risk of depression recurrence.

The present invention will be further illustrated below with reference to the specific examples. It should be understood that these examples are only for illustrating the present invention and not intend to limit the scope of the present invention. Experimental methods not specified in the following examples are usually under conventional conditions, such as conditions described in Sambrook et al., Molecular Cloning: Laboratory Manual (New York: Cold Spring Harbor Laboratory Press, 1989), or as recommended by the manufacturer. Unless otherwise specified, percentages and portions are weight percentages and weight portions.

### General Methods

### Real Time Fluorescence Quantitative PCR

Real time fluorescence quantitative PCR is an experimental method that applies fluorescence energy technology to polymerase chain reaction. A fluorescent dye called SYBR Green I will be used in this experiment. In the PCR reaction system, SYBR Green I is specifically incorporated into DNA double stranded and emits a fluorescence signal; while the SYBR dye molecules that are not incorporated into the chain will not emit any fluorescence signals. Since this method keeps the increase in fluorescence signal synchronized with the increase in PCR products, that is, the intensity of fluorescence signal emitted by fluorescent dyes is directly proportional to DNA production. Therefore, by detecting the fluorescence signal intensity of the PCR process, the initial concentration of the target sequence can be determined, thus achieving the purpose of quantitative analysis.

### Example 1

The subjects were 20 first-onset and recurrent depression patients included in the Affiliated Kangning Hospital of Ningbo University and the Ningbo Mental Health Center, and 20 age and gender matched healthy volunteers were recruited as controls. All physicians involved in the diagnostic work have the qualification of practicing psychiatrists and have more than 10 years of experience in psychiatry. They are proficient in using the SCID-1 checklist and are proficient in the ICD-10 and DSM-V diagnostic criteria. They used the Hamilton Depression Scale (HAMD-17) to evaluate the patient's psychopathological state, under standardized and consistent procedures. The consistency test met the requirements (Kappa-0.68-0.82), and depression patients with comorbidities were excluded. This study was approved by the Ethics Committee of Ningbo University, and all study subjects signed informed consent forms. The next morning after the subjects were enrolled, 20 ml of venous blood was drawn on an empty stomach for scientific research.

Diagnosis, inclusion, and exclusion criteria for patients with recurrent depression, first-onset depression, and healthy controls:

Group of patients with first-onset depression: (1) inclusion criteria: all cases met the diagnostic criteria for depressive episodes (F32) in the International Classification of Mental and Behavioral Disorders (ICD-10), and the episode was regarded as the first onset; (2) statistics: Han ethnicity, age 18-60 years old, primary school education or above, gender distribution, disease course, medication use, etc.; (3) the score of Hamilton Depression Scale (17 items, HAMD-17) was greater than or equal to 17 points; (4) the patients or their legal guardians signed an informed consent form; (5) all patients were included in the Affiliated Kangning Hospital of Ningbo University and the Ningbo Mental Health Center. (6) Exclusion criteria: same as the group of patients with recurrent depression below.

Group of patients with recurrent depression: (1) inclusion criteria: all cases met the diagnostic criteria for recurrent depression (F33) in the International Classification of Mental and Behavioral Disorders (ICD-10), with at least one previous depressive episode; (2) statistics: Han ethnicity, age 18-60 years old, primary school education or above, gender distribution, disease course, medication use, etc.; (3) the score of Hamilton Depression Scale (17 items, HAMD-17) was greater than or equal to 17 points; (4) the patients or their legal guardians signed an informed consent form; (5) all patients were included in the Affiliated Kangning Hospital of Ningbo University and the Ningbo Mental Health Center, with complete medical records such as “first visit records” and “discharge records”; (6) exclusion criteria: those with current or past other mental disorders; combining severe or chronic physical diseases and organic brain diseases (neurodegenerative diseases, traumatic brain injury, or cerebrovascular diseases); users of psychoactive substances; pregnant and lactating women; individuals with communication difficulties; uncooperative individuals; multiple episodes of depression without complete records due to outpatient or inpatient visits to other general hospitals; all the above subjects would be excluded.

Group of healthy control: (1) inclusion criteria: no previous or current mental illness and clear physical illness; no family history of mental illness; (2) statistics: Han ethnicity, age 18-60 years old, primary school education or above, gender distribution, disease course, medication use, etc.; (3) the score of Hamilton Depression Scale (17 items, HAMD-17) was less than 7 points; (4) exclusion criteria: same as the group of patients with recurrent depression and the group of patients with first-onset depression mentioned above.

1. Analysis of HIF-1α levels in peripheral blood of first-onset depression patients, recurrent depression patients, and healthy control patients.

Approximately 4 ml of peripheral blood from each subject was collected and subjected to clot at room temperature for 1 hour, then the sample was centrifuged at 3000×g for 10 minutes to obtain serum. Then the serum was stored in a low-temperature refrigerator at −80° C. or analyzed directly. An ELISA kit was constructed for detecting HIF-1α protein in peripheral blood of first-onset depression patients, recurrent depression patients, and healthy control patients.

The concentration of serum HIF-1α protein was detected using an ELISA kit, which provided necessary but not all reagents or materials for detecting HIF-1α protein concentration: one 96 well ELISA plate (which has been coated with recombinant human HIF-1α protein specific antibodies), two tubes of human HIF-1α protein standards, one bottle of 12 ml experimental diluent RD1-38, one bottle of 21 ml human HIF-1α protein conjugated substrate solution, one bottle of 21 ml calibration diluent RD5P, one bottle of 21 ml washing solution (25 fold dilution), one bottle of 12 ml chromogenic solution A, one bottle of 12 ml chromogenic solution B, and one bottle of 6 mL termination solution. In addition, additional reagents and materials were required as follows: enzyme-linked immunosorbent assay (ELISA) reader, pipettor, pipette, graduated cylinder, absorbent paper, distilled or deionized water, data analysis and plotting software, etc.

All necessary items were prepared before testing began:
\textcircled{1} Washing solution preparation: 20 mL of washing solution was taken and deionized water was added to dilute it to 500 mL for later use;
\textcircled{2} Substrate solution: an equal volume of chromogenic solution A and B were mixed 15 minutes before use;
\textcircled{3} The calibration diluent RD5P was diluted by adding 20 mL of RD5P stock solution to 80 ml of deionized water and mixing it to form 100 mL of the diluted calibration diluent RD5P for later use;
\textcircled{4} HIF-1α protein standard preparation: diluted calibration diluent RD5P was added to one tube of HIF-1α protein standard to construct a series of gradient concentration standards. The concentrations of standards are 100 ng/ml, 50 ng/ml, 25 ng/mL, and 6.25 ng/ml, respectively.

The experimental operations were as follows:
\textcircled{1} the 96 well enzyme-linked immunosorbent assay (ELISA) plate provided in the kit (which has been coated with recombinant human HIF-1α protein specific antibodies) was used, and the number of wells required for this test was determined. Duplicate wells should be set for each sample, standard and blank. In addition, according to one's own needs, conventional immune antibody preparation methods could be used, by injecting purified HIF-1α protein or its antigen fragments into animal bodies to produce specific antibodies, or immunizing animals with cells expressing human HIF-1α protein or its antigen fragments to produce antibodies, and the antibodies were coated in a clean enzyme-linked immunosorbent assay plate, thereby a new kit could be made.
\textcircled{2} 50 μl of test diluent RD1-38 was added to each well first;
\textcircled{3} Samples addition: after diluting the HIF-1α protein standard by the double-dilution method, 50 μL each of 100 ng/mL, 50 ng/mL, 25 ng/ml, and 6.25 ng/mL standards were added to the wells of the enzyme-linked immunosorbent assay (ELISA) plate in sequence, and a blank comparison well was set up (no sample or ELISA reagent was added to the blank comparison well, and the other steps were the same). 50 μL of the serum sample to be tested was added to the remaining wells after dilution with calibration diluent RD5P (the concentration of HIF-1α protein in the diluted serum was in the middle of the concentration range shown by the standard should be ensured and the dilution ratio of each sample should be recorded for calculation after the experiment to ensure the accuracy of the detection by the kit); The plate was incubated on a shaker for 2 hours at room temperature; The shaking speed was set to 500±50 rpm;
\textcircled{4} The liquid inside the well was poured out completely, and each well was washed 4 times with the diluted washing solution, with approximate 400 μl of washing solution was added in each well. After washing, the plate was inverted onto an absorbent paper and patted dry;
\textcircled{5} 200 μl of human HIF-1α protein conjugated substrate solution was added to each well and incubated on a shaker for 1 hour at room temperature;
\textcircled{6} the washing step of (4) was repeated;
\textcircled{7} 200 μl of substrate solution was added to each well and incubated in dark for 30 minutes at room temperature;
\textcircled{8} 50 μl of termination solution was added to each well to stop the reaction (the color immediately changed from blue to yellow);
\textcircled{9} The optical density (OD value) was measured at 450 nm using an enzyme-linked immunosorbent assay (ELISA) reader within 15 minutes after adding the termination solution, and the blank hole was set as zero. The OD values measured using the standard protein of HIF-1α were plotted as a standard curve using ELISACalc software, along with the mathematical formulas for the curve. Then, the OD values of each serum sample were sequentially input into the standard curve formula obtained by the software to calculate the content of HIF-1α protein in the current sample. Based on the dilution ratios recorded before the experiment, the undiluted concentration was calculated as the final content of HIF-1α protein in the serum.

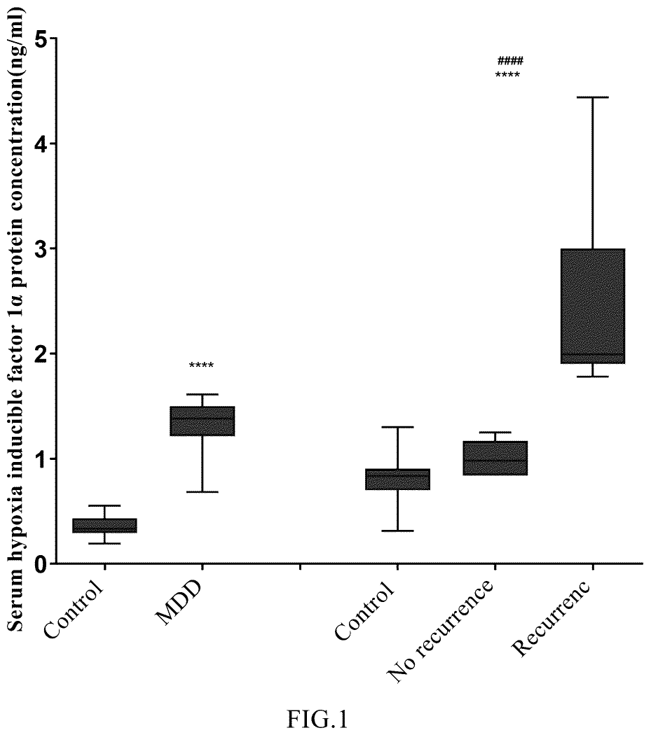

As shown in FIG. 1, the serum levels of HIF-1α protein were significantly increased only in patients with recurrent depression, and there was no statistically significant difference between healthy subjects and those who did not experience depression recurrence after drug treatment. This indicates that the detection of HIF-1α protein content in the present invention can effectively and specifically screen or differentiate depression recurrence in the early stage, and can also be used as a biomarker for evaluating the efficacy of intervention treatment in depression patients. In addition, some other enzyme-linked immunosorbent assay kits that can detect hypoxia inducible factor 1α have the same effect as the kit used in the above example, such as HIF1A (Human) ELISA Kit (RAB0507, Abnova), human HIF-1 alpha ELISA Kit (ab171577, Abcam), etc.

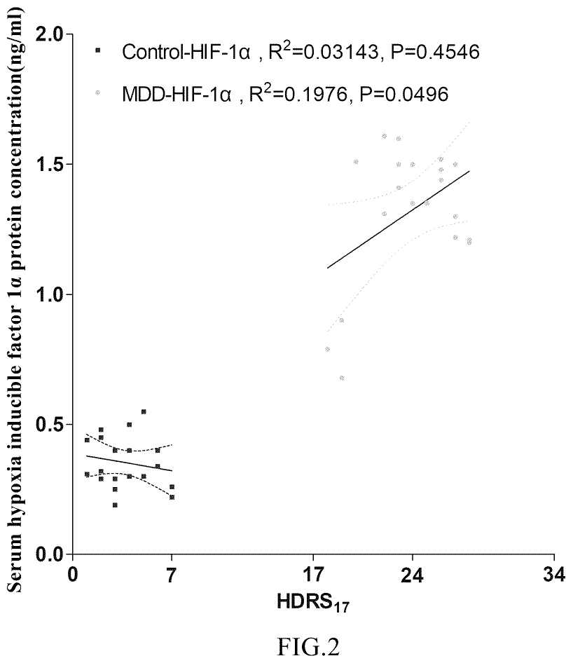

FIG. 2 shows the HAMD-17 scores and the correlation analysis of HIF-1α expression in peripheral blood of healthy and depressed subjects in Example 1 of the present invention.

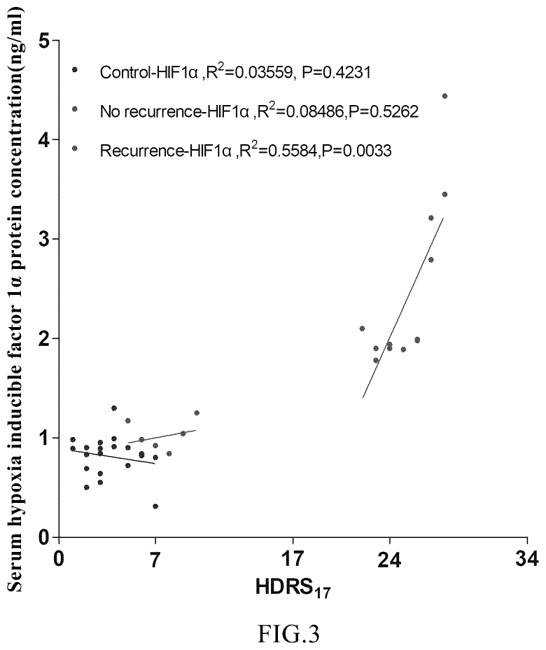

FIG. 3 shows the HAMD-17 scores and the correlation analysis of HIF-1α expression in peripheral blood of healthy, recurrent and non-recurrent depression subjects after treatment in Example 1 of the present invention.

2. Correlation analysis of HIF-1α content in peripheral blood of first-onset depression patients, recurrent depression patients, and healthy control subjects with the Hamilton Depression Scale HAMD-17. The test results are shown in Table 1.

TABLE 1
Basic clinical information of subjects and HAMD-17 test results

| | Groups | | |
|---|---|---|---|
| | Healthy | Depressed | |
| | subjects | patients | |
| Observation indicators | Control | MDD | P-value |
| Number of participants | 20 | 20 | |
| Gender (male/female) | (10/10) | (10/10) | |
| Symptom Assessment - | 3.500 ± 0.3285 | 23.65 ± 0.553 | <0.0001 |
| HAMD-17 | | | |

On the basis of the analysis of the HIF-1α content in the peripheral blood of the subjects, the Hamilton Depression Scale (HDRS-17) was collected, and the correlation analysis was conducted between the HDRS-17 scores and the HIF-1α expression levels in the peripheral blood of healthy subjects (Control, n=20) and depressed patients (MDD, n=20).

TABLE 2
Correlation analysis between HAMD-17 score and peripheral
blood HIF-1α expression in healthy and depressed subjects

| | Groups | |
|---|---|---|
| | Healthy | Depressed |
| | subjects | patients |
| Observation indicators | Control(n = 20) | MDD(n = 20) |
| No depression symptoms (<7) | 0.367 ± 0.092 | NA(n = 0) |
| | (n = 18) | |
| Mild or moderate depression symptoms | 0.240 ± 0.020 | 1.287 ± 0.320 |
| (≥17 and ≤24) | (n = 2) | (n = 11) |
| Severe depression symptoms (>24) | NA(n = 0) | 1.358 ± 0.124 |
| | | (n = 9) |

On the basis of the analysis of the HIF-1α content in the peripheral blood of the subjects, the Hamilton Depression Scale (HDRS-17) was collected, and the correlation analysis was conducted between the HDRS-17 scores and the expression levels of HIF-1α in the peripheral blood of healthy subjects (Control, n=20), non-recurrent patients after drug treatment in first-onset severe depression (No recurrence, n=8), and recurrent patients after drug treatment in first-onset severe depression (Recurrence, n=12).

TABLE 3
Correlation analysis between HAMD-17 scores and HIF-1α expression
in peripheral blood of healthy subjects, recurrent depression subjects
after treatment, and non-recurrent depression subjects after treatment

| | Groups | | |
|---|---|---|---|
| | | Non-recurrent | |
| Observation | Healthy subjects | depression patients | Recurrent depression |
| indicators | Control(n = 20) | (n = 20) | patients (n = 12) |
| No depression symptoms (<7) | 0.841 ± 0.176(n = 18) | 0.997 ± 0.135(n = 4) | NA(n = 0) |
| Mild or moderate depression | 0.555 ± 0.245(n = 2) | 1.013 ± 0.154(n = 4) | 1.924 ± 0.103(n = 4) |
| symptoms (≥17 and ≤24) | | | |
| Severe depression symptoms | NA(n = 0) | NA(n = 0) | 2.718 ± 0.869(n = 8) |
| (>24) | | | |

Conclusion: The changes in HIF-1α in peripheral blood of first-onset severe depression patients and the recurrent and non-recurrent cases after drug treatment were found to be significantly higher in severe depression compared to the control group. After drug treatment, the recurrent group showed a significant increase in HIF-1α compared to the non-recurrent group. Through correlation analysis, it was found that HIF-1α was correlated with the recurrence of depression. Based on the clinical cases, it has been preliminarily found that HIF-1α serves as a biomarker for depression recurrence.

### Example 2

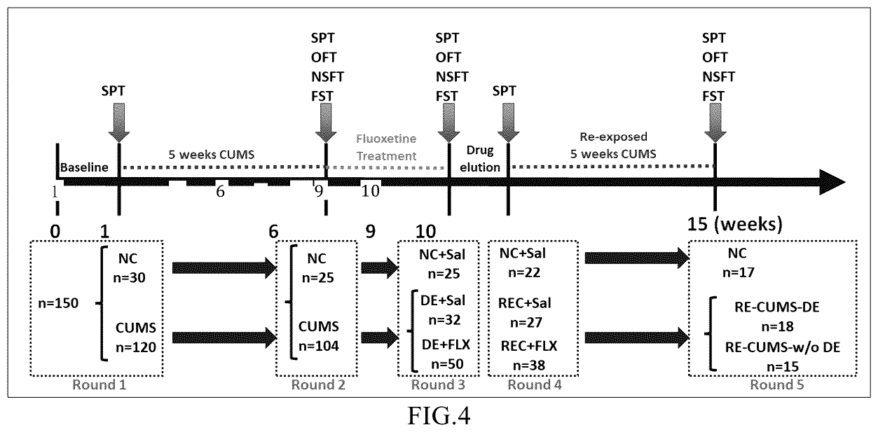

Construction of a chronic unpredictable mild stress (CUMS) induced depression recurrence model and evaluation of related depression like behavior and brain pathological changes: 150 C57BL/6 male adult mice were selected for model construction, and the CUMS model was subjected to stress for 5 weeks each time. After one week of adaptation to the environment, SPT behavioral assessment was conducted to screen healthy male mice with normal behavioral performance. Firstly, the mice were randomly divided into a control group and an experimental group. The control group mice were fed normally, while the experimental group mice were given 5 weeks of CUMS stimulation. After 5 weeks of chronic stress, depression like mice and control group mice were selected. Then, the depression like mice were divided into two groups: one group was given Fluoxetine (20 mg/kg/day), and the other group and control group were given the same dose of physiological saline. After one week of drug elution, mice recovered from fluoxetine treatment were given another 5 weeks of CUMS. After another 5 weeks of chronic stress, depression like mice and non depression like mice after another CUMS were selected, as well as control group mice. The three groups of mice were studied (FIG. 4).

The CUMS method used in this experiment includes: cold water swimming (4° C., 5 minutes), hot water swimming (42° C., 5 minutes), tilted rat cage (tilted 45° C., 24 hours), paired feeding (24 hours), fasting (24 hours), water deprivation (24 hours), day night reversal (24 hours), continuous lighting (36 hours), moist rat cage (24 hours, with appropriate water added to the cage). A total of nine stimuli were randomly arranged to be completed within 4 weeks, and the same stimulus cannot appear continuously, making it impossible for animals to predict the occurrence of the stimulus. The experimental group was raised in a single cage, while the control group was raised in a normal manner.

Administration method: before each administration, fluoxetine hydrochloride capsules were dissolved in physiological saline to prepare a fluoxetine solution of 10 mg/ml. During the administration period, mice were given fluoxetine (10 mg/kg) or the same dose of physiological saline by gavage daily for 3 weeks.

Drug elution period: it is known that the metabolic clearance half-life of fluoxetine in humans is 4-6 days, while in mice, the clearance half-life of fluoxetine is 9 hours. However, other references have reported that the half-life of fluoxetine clearance in mice is various, and the drug elution cycle in this experiment is one week.

Animal behavioral assessment: before the start of the experiment, after the first 4 weeks of CUMS, after the recovery of depression like mice induced by the first CUMS, and after the second 4 weeks of exposure to CUMS, corresponding behavioral assessments were conducted, including: (1) Sugar Preference Test (SPT); (2) Open field test (OFT); (3) Novelty Suppressed Feeding Test (NSFT); (4) Forced Swimming Test (FST), with experimental methods following standard operating procedures.

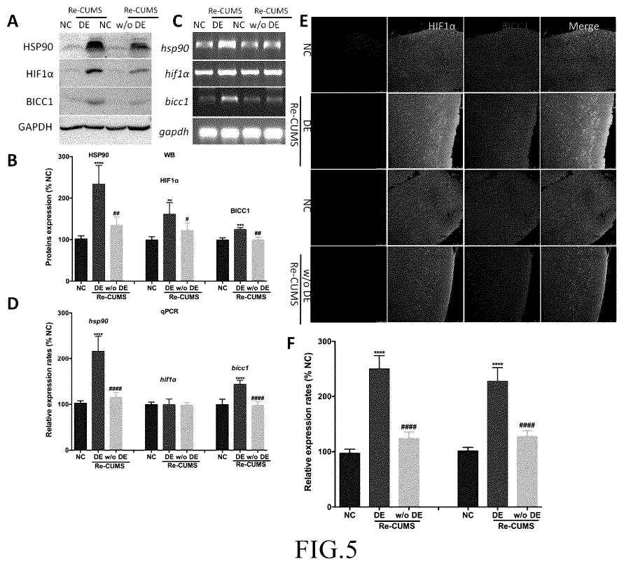

After conducting depression like behavior tests and through sugar water preference test (SPT), open field test (OFT), novelty suppressed feeding test (NSFT), and forced swimming test (FST) and screening of mice, 17 mice were ultimately included in the control group (NC), 18 mice in the secondary stress depression group (RE-CUMS-DE), and 15 mice in the secondary stress non depression group (RE-CUMS-w/o DE). Western Blot, Realtime PCR, and immunohistochemistry experiments were used to evaluate the expression and transcription changes of HSP90, HIF-1α, and BICC1 in the prefrontal cortex of mice with recurrent depression (FIG. 5).

Conclusion: The Western blot results indicated that RE-CUMS significantly induced an increase in HSP90, HIF-1α, and BICC1 protein expression. The Realtime PCR results showed that RE-CUMS significantly induced an increase in hsp90 and bicc1 transcription levels, while hif-1α did not show a significant increase. The situation where HIF-1 alpha protein is elevated but mRNA is not elevated is often accompanied by an increase in HSP90 protein level, indicating that the decrease in HIF-1 alpha degradation is related to HSP90 protein. The immunohistochemical study results showed that RE-CUMS significantly induced an increase in HSP90 and BICC1 labeled positive neurons. The situation where the expression level or activity of HIF-1 alpha protein is elevated but mRNA is not elevated can be used for the evaluation of depression recurrence, including but not limited to changes in the genome, transcriptome, and proteome levels of HSP90 and BICC1.

### Discussion

Hypoxia inducible factor-1 (HIF-1) is a heterodimeric molecule, in which the functionally active subunit is Hypoxia inducible factor-1 alpha (HIF-1α), which is a core factor involved in host cell hypoxia adaptation regulation. When HIF-1α is activated, it participates in: (1) tissue recovery after hypoxia; (2) pro-inflammatory and antimicrobial effects; (3) promoting tumor growth; (4) specific pro-apoptotic effect; (5) regulating embryonic development and other various physiological processes.

The inventor unexpectedly found through research that hypoxia inducible factor 1 alpha (HIF-1α) is significantly correlated with the recurrence of depression. Due to the low compliance and severe injury of HIF-1α detection in the brain or cerebrospinal fluid of patients with depression, it is difficult to apply in clinical practice.

In the present invention, by selecting peripheral blood HIF-1α (combined with HSP90, BICC1, etc.) as a biomarker, the diagnosis of the condition of recurrent depression patients can be achieved, and an objective diagnostic method for depression recurrence can be achieved, with high sensitivity and specificity for depression diagnosis.

On the other hand, by constructing a mouse model of depression recurrence induced by chronic unpredictable mild stress, and evaluating related depressive like behaviors and brain pathological changes, it has been determined that the peripheral blood HIF-1α protein can serve as a key biomarker for warning or diagnosing depression recurrence. Gene screening and protein expression level detection can be used to determine the potential risk or progression of depression recurrence.

Since the method of the present invention uses peripheral blood or serum samples as detection samples, the method is easy to obtain and has high patient compliance, and helps to solve the problems of early warning and recurrence diagnosis of depression.

All references mentioned in the present application are incorporated by reference herein, as though individually incorporated by reference. In addition, it should be understood that after reading the above teaching content of the present invention, various changes or modifications may be made by those skilled in the art, and these equivalents also fall within the scope as defined by the appended claims of the present application.

1-15. (canceled)

16. A method for diagnosing a risk of depression recurrence or evaluating a therapeutic effect of depression recurrence, which comprises:
detecting a level of a depression recurrence risk marker in a tested sample; wherein, the depression recurrence risk marker comprises HIF-1α protein.

17. The method of claim 16, wherein the tested sample is selected from the group consisting of blood, plasma, serum, and a combination thereof.

18. The method of claim 16, wherein the depression recurrence risk marker further comprises a gene, transcript, or protein of one or more markers selected from the group B consisting of (B1) HSP90 and (B2) BICC1.

19. The method of claim 16, wherein when the expression level C1 of the risk marker HIF-1α protein of a subject is significantly higher than the reference value C0, it indicates that the subject has a high risk of depression recurrence.

20. The method of claim 16, wherein if (a) the protein quantity or activity of HIF-1α increases in the tested sample of a subject but there is no significant change in mRNA level of HIF-1α, and (b) the HSP90 protein quantity or activity or mRNA level increases, it indicates that the subject has an increased risk of depression recurrence.

21. The method of claim 16, wherein if (a) the protein quantity or activity of HIF-1α increases in the tested sample of a subject, and (b) the protein quantity or activity or mRNA level of HSP90 increases, it indicates that the subject has an increased risk of depression recurrence.

22. The method of claim 16, wherein if (a) the protein quantity or activity or mRNA level of BICC1 increases in the tested sample of a subject, and (b) the protein quantity or activity of HIF-1α increases, it indicates that the subject has an increased risk of depression recurrence.

23. The method of claim 16, wherein the tested sample is derived from a subject selected from the group consisting of: a subject without depression, a subject which is susceptible to depression, a patient with first-onset depression, a patient with depression recurrence, and a combination thereof.

24. A depression recurrence risk assessment device, which comprises:
(a) an input module, which is configured for inputting depression recurrence risk marker data in blood, plasma, or serum of a subject;
wherein, the risk marker comprises HIF-1α protein;
(b) a data processing module, which is configured for processing depression recurrence risk marker data and providing recurrence risk assessment result, wherein the processing comprises: comparing the expression level C1 of the marker with the reference value C0, wherein, when C1 is significantly higher than C0, it indicates that the subject has a high risk of depression recurrence; when C1 is not significantly higher than C0, it indicates that the subject has a low risk of depression recurrence; and
(c) an output module, which is configured for outputting the assessment result.

25. The device of claim 24, wherein the device further comprises a detection module, which is configured for detecting protein level or activity of the risk marker.

26. The device of claim 25, wherein the detection module is selected from the group consisting of: an ELISA analyzer, a PCR instrument, a sequencer, and a combination thereof.
